(19) United States
(12) Patent Application Publication
(10) Pub. No.: US 2026/0224604 A1
(43) Pub. Date: Aug. 6, 2026

(54) POLYNUCLEOTIDES AND USE OF SAME FOR WOUND HEALING

(71) Applicant: RAMBAM MED-TECH LTD., Haifa (IL)

(72) Inventors: Mogher KHAMAISI, Haifa (IL); Salim HADAD, Haifa (IL)

(73) Assignee: RAMBAM MED-TECH LTD., Haifa (IL)

(21) Appl. No.: 19/150,586

(22) PCT Filed: Jan. 24, 2024
(86) PCT No.: PCT/IL2024/050095

§ 371 Date: Jul. 24, 2025

Related U.S. Application Data
(60) Provisional application No. 63/440,759, filed on Jan. 24, 2023.

(51) Int. Cl. A61K 31/7105; A61K 35/33; A61P 17/02; C12N 5/077; C12N 15/113

(52) U.S. Cl. CPC A61K 31/7105; A61K 35/33; A61P 17/02; C12N 5/0656; C12N 15/113; C12N 2310/141

(57) ABSTRACT

Methods for treating a wound in a subject in need thereof, by a miR molecule, or by a fibroblast expressing the miR molecule, from the miR-1 family, the precursor thereof, or the mature product thereof, are provided. Further provided are pharmaceutical compositions including the miR molecule, or a fibroblast expressing the miR molecule, selected from hsa-miR-1-3p, hsa-mir-1-1, and hsa-miR-1-2, for use in treatment of a wound in a subject in need thereof.

### CROSS-REFERENCE TO RELATED APPLICATIONS

This application claims the benefit of priority of U.S. Provisional Patent Application No. 63/440,759, titled “POLYNUCLEOTIDES AND USE OF SAME FOR WOUND HEALING”, filed Jan. 24, 2023. The contents of all the above applications are incorporated herein by reference in their entirety.

### REFERENCE TO AN ELECTRONIC SEQUENCE LISTING

The contents of the electronic sequence listing (TRMB-P-028-PCT.xml; size: 3,752 bytes; and date of creation: Jan. 1, 2024) are herein incorporated by reference in their entirety.

### FIELD OF INVENTION

The present invention is in the field of wound healing and metabolic diseases.

### BACKGROUND

Diabetes mellitus (DM) associated complications pose a major health-care burden worldwide and present major challenges to patients, health-care systems, and national economies. Despite the prevalence and the debilitating nature of diabetic vascular complications, the cellular, molecular, genetic, and epigenetic mechanisms underlying vascular dysfunction remain unclear. Chronic foot ulcers are one of the most common and serious complications of diabetes. Peripheral vascular disease, neuropathy, insulin resistance, and reduced resistance to infection related to poor glycemic control are recognized risk factors for poor wound healing in individuals with diabetes. Indeed, diabetes is associated with impaired wound healing, making these patients susceptible to chronic non-healing wounds.

Wound healing process is known to results from the complex biological and molecular events of angiogenesis, cell adhesion, migration, proliferation, differentiation, and extracellular matrix (ECM) deposition. Wounds normally heal in a very orderly and efficient manner characterized by four distinct, but overlapping phases, comprising hemostasis, inflammation, proliferation and remodeling. Lack of endothelial regeneration and impaired angiogenesis contribute to the progression of diabetic microvascular and macrovascular complications. The identification of mechanisms and therapeutic targets that contribute to poor wound healing in diabetes has been challenging and still remain unclear.

Multiple treatment modalities using cytokine replacement and transplantation of keratinocytes or fibroblasts are effective in non-diabetic populations but their efficacy in patients with diabetes is diminished due to undetermined mechanisms. Aggressive antibiotic therapy with surgical debridement, along with use of negative pressure wound therapy, is still considered the best option for improving wound healing in diabetic patients.

There is still a great need for treatments enhancing or improving wound healing processes, primarily, such as, for the improvement of both quality- and duration-life of diabetic patients afflicted with wounds.

### SUMMARY

The present invention, in some embodiments, is based on the surprising finding that the level of a micro-RNA (miR) molecule, from the Homo sapiens-(hsa-) miR-1 family, comprising hsa-miR-1-3p, is significantly reduced in a fibroblast from a diabetic patient (FIGS. 2B and 3). Moreover, it was surprisingly found that a diabetic fibroblast expressing hsa-miR-1-3p, for example by transfection with hsa-miR-1-3p mimics, demonstrated enhanced migratory ability (FIG. 5) and increased vascular endothelial growth factor (VEGF) protein expression (FIG. 7) and secretion (FIG. 6).

According to one aspect, there is provided a method of treating a wound in a subject in need thereof, the method comprising administering to the subject a therapeutically effective amount of a pharmaceutical composition comprising an agent capable of increasing expression, abundance, or both, of a micro-RNA (miR) molecule belonging to miR-1 family in the subject, thereby treating a wound in the subject.

According to another aspect, there is provided a method for treating a wound in a subject in a need thereof, the method comprising the steps of: (a) providing a composition comprising a fibroblast being characterized by increased expression, abundance, or both, of a miR molecule from miR-1 family; and, (b) administering a therapeutically effective amount of the composition from step (a) to the subject, thereby treating a wound in the subject.

According to another aspect, there is provided pharmaceutical composition comprising a nucleic acid molecule being a miR molecule belonging to miR-1 family, for use in the treatment of a wound in a subject in need thereof.

According to another aspect, there is provided a pharmaceutical composition comprising a modified fibroblast being characterized by increased expression, abundance, or both, of a miR molecule belonging to miR-1 family, compared to a control fibroblast.

In some embodiments, the agent is selected from the group consisting of: a miR molecule, a precursor thereof, and a combination thereof.

In some embodiments, the pharmaceutical composition further comprises any one of: a liposome, a carrier, and both.

In some embodiments, any one of the: miR molecule, precursor thereof, and combination thereof, is encapsulated within the liposome.

In some embodiments, any one of the: miR molecule, precursor thereof, and combination thereof, is bound to the carrier.

In some embodiments, the carrier comprises chitosan.

In some embodiments, the method further comprises a step preceding the step (a) comprising obtaining a fibroblast and contacting the fibroblast with an agent capable of increasing expression, abundance, or both, of a miR molecule belonging to miR-1 family, in the fibroblast.

In some embodiments, the fibroblast is obtained or derived from the subject.

In some embodiments, the subject is afflicted with diabetes.

In some embodiments, the agent is selected from the group consisting of: miR molecule, a precursor thereof, and a combination thereof.

In some embodiments, increased is compared to a control fibroblast.

In some embodiments, the miR molecule belonging to miR-1 family comprises a nucleic acid sequence being selected from the group consisting of: TGGAATGTAAAGAAGTATGTAT (SEQ ID NO: 1) or a functional analog thereof having at least 80% sequence homology thereto, TGGGAAACATACTTCTTTATATGCCCATATGGACCTGCTAAGCTATGGAATGTAAA GAAGTATGTATCTCA (SEQ ID NO: 2) or a functional analog thereof having at least 80% sequence homology thereto, and ACCTACTCAGAGTACATACTTCTTTATGTACCCATATGAACATACAATGCTATGGA ATGTAAAGAAGTATGTATTTTTGGTAGGC (SEQ ID NO: 3) or a functional analog thereof having at least 80% sequence homology thereto, and any combination thereof.

In some embodiments, the miR molecule comprises the nucleic acid sequence set forth in SEQ ID NO: 1, or a functional analog thereof having at least 80% sequence homology thereto.

In some embodiments, the subject is afflicted with a disease or disorder selected from the group consisting of: metabolic disease or disorder, anemia, arterial circulatory disorder, gout, chronic venous insufficiency, rheumatoid arthritis, lupus, scleroderma, and any combination thereof.

In some embodiments, the metabolic disease or disorder is selected from the group consisting of: pre-diabetes, type 1 diabetes, type 2 diabetes, insulin resistance or insulin-resistance related, gestational diabetes, hyperglycemia, and any combination thereof.

In some embodiments, the administering comprises locally administering the pharmaceutical composition to the wound of the subject.

In some embodiments, the miR molecule belonging to miR-1 family comprises a mature miR molecule, a precursor thereof, or a combination thereof.

In some embodiments, the mature miR molecule or precursor thereof comprises a nucleic acid sequence being selected from the group consisting of: SEQ ID NO: 1 or a functional analog thereof having at least 80% sequence homology thereto, SEQ ID NO: 2 or a functional analog thereof having at least 80% sequence homology thereto, SEQ ID NO: 3 or a functional analog thereof having at least 80% sequence homology thereto, and any combination thereof.

In some embodiments, the mature miR molecule comprises the nucleic acid sequence set forth in SEQ ID NO: 1.

In some embodiments, the modified fibroblast is obtained by contacting a fibroblast derived or obtained from a diabetic subject with an agent capable of increasing expression, abundance, or both, of a miR molecule belonging to miR-1 family.

In some embodiments, the pharmaceutical composition is for use in treating a wound in a subject in need thereof.

In some embodiments, the pharmaceutical composition is formulated for local administration.

In some embodiments, local administration comprises administration directedly to the wound of the subject.

In some embodiments, the pharmaceutical composition is in form of: a suspension, a spray, a cream, an ointment, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B includes images and histograms demonstrating Fibroblast's morphology and cell surface marker. (1A) Morphological images of control (non-diabetic) and diabetic fibroblasts taken by light microscopy. The microscopic pictures were taken at ×10 magnification. (1B) Flow cytometric analysis of cell surface marker CD90. More than 75% of the cells expressed CD90.

FIGS. 2A-2C include images and graphs demonstrating miRNA profile of diabetic fibroblasts (DF) that are differentially expressed compared to non-diabetic fibroblasts (NDF). (2A) In principle component analysis (PCA) the different samples were not found to be clustered according to condition, diabetic vs. non-diabetic. (2B) Normalized miRNA expression levels in DF vs NDF. Green and red colors represent up- and down-regulated miRNAs in DF respectively (fold change >2 or <−2, P<0.05), black dots represent non-significant differentially expressed miRNAs. (2C) A scheme showing the predicted regulatory effects of hsa-miR-1-3p in signaling pathways constructed by QIAGEN Ingenuity Pathway Analysis.

FIG. 3 includes a bar graph demonstrating RT-PCR quantification presented in logarithmic scale. Downregulation of hsa-miR-1-3p in diabetic fibroblasts was observed. * P<0.05.

FIGS. 4A-4B include bar graphs and images showing the transfection efficacy of hsa-miR-1-3p. (4A) Diabetic fibroblasts cell culture was transfected with hsa-miR-1-3p and negative control (NC) miRNA mimic, for a period of 24, 48 and 72 hours. The cells were then extracted with the mirvana miRNA isolation kit then quantified for PTK9 using the TaqMan PCR assay, and by using GAPDH as an internal, housekeeping control gene. qRT-PCR revealed efficiency of transfection of hsa-miR-1-3p mimic into diabetic fibroblasts, compared to the negative control miRNA, as demonstrated by PTK9 downregulation. Data were analyzed with the student's t-test and are presented as the mean±standard error of the mean. Experiment was carried out in triplicate. (4B) The BLOCK-iT™ Alexa Fluor® Red Fluorescent Control was transfected into diabetic fibroblasts using Lipofectamine RNAiMAX Transfection Reagent. Final oligo concentration was 50 nM. 24 hours after the start of transfection, growth medium was removed and replaced with PBS. Nuclear localization of the oligo is seen by florescence microscope. Almost 100% of the cells took up the oligo and cells retain a normal morphology as seen in the phase images. * P<0.05.

FIG. 5 includes bar graphs and images showing wound closure of diabetic fibroblasts in a scratch migration assay. The graph represents migration results of fibroblasts from 3 diabetic donors. Representative images of scratch assay at 4 time points post scratch initiation. Data is presented as mean±SEM of relative wound density percentage. * P<0.05, NC; negative control.

FIG. 6 includes bar graphs showing the effect of hsa-miR-1-3p expression on VEGF secretion from diabetic fibroblasts. Data represents quantitative relation of basal VEGF levels from ELISA experiments normalized to total protein mass. Cells were treated with 100 nM insulin for 24 hours as a positive control. Fibroblasts were obtained from 3 diabetic donors. Squared is a graph representing averaged VEGF secreted levels from the 3 diabetic donors. Data presented as mean±SEM. * P<0.05.

FIG. 7 includes images and graphs showing the effect of hsa-miR-1-3p on different proteins expression. Representative immunoblots from one donor (T2DM). Relative quantification graph of VEGF, FGF-2 and N-cadherin expression levels normalized to GAPDH. Insulin treatment served as positive control. UNT, untreated; N-cad, N-cadherin.

FIGS. 8A-8B include micrographs and a graph showing that has-miR-1-3p accelerates wound closure in vivo. (8A) Micrographs of wound area from diabetic and non-diabetic rats taken at day 0, 4, 6, and 8 of treatment with: has miR-p-1-3p, untreated, or negative control (NC). (8B) A graph presenting wound closure rate of the treatment groups presented in 8A. Wound closure % was calculated according to the following: Percentage Closed (y %)=[(Area on Day 0-Open Area on Day X)/Area on Day 0]×100

### DETAILED DESCRIPTION

According to one aspect, there is provided a method for treating a PTK9 (encoding Twinfilin Actin Binding Protein 1 (PTK)-related disease in a subject in need thereof. According to some there is provided a method for treating a phosphoinositide 3-kinase (PIK3C2A)-related disease in a subject in need thereof. In some embodiments, the method comprises administering to the subject a therapeutically effective amount of a pharmaceutical composition comprising a micro-RNA (miR) molecule belonging to miR-1 family or a precursor thereof.

As used herein, the terms “PTK-related disease” or “PIK3C2A-related disease” refers to any disease, condition, disorder, pathology, or any combination thereof, wherein a PTK or PIK3C2A gene or a protein encoded therefrom is involved, induces, initiates, propagates, determines, or any combination or equivalent thereof, in the pathogenesis, pathophysiology, or both, respectively.

### Methods and Use Thereof

In some embodiments, there is provided a method of treating a wound in a subject in need thereof, the method comprising administering to the subject a therapeutically effective amount of a pharmaceutical composition comprising an agent capable of increasing expression, abundance, or both, of a miR molecule belonging to miR-1 family in the subject, thereby treating a wound in the subject.

In some embodiments, there is provided a method of treating a wound in a subject in need thereof, the method comprising administering to the subject a therapeutically effective amount of a pharmaceutical composition comprising a nucleic acid molecule capable of increasing expression, abundance, or both, of a miR molecule belonging to miR-1 family in the subject, thereby treating a wound in the subject.

In some embodiments, the agent comprises a nucleic acid molecule.

In some embodiments, the agent comprises micro-RNA (miR) molecule belonging to miR-1 family or a precursor thereof.

In some embodiments, treating a wound comprises improving or ameliorating the healing of a wound.

As used herein, the term “wound healing” comprises replacement damaged tissue by a newly produced tissue in a subject. A normal wound healing process comprises a regulated sequence of biochemical events, required to repair a damage in subject's skin. In some embodiments, a wound comprises skin damage in the epidermis, dermis, or both, in a subject. In some embodiments, wound healing comprises blood clotting or hemostasis, inflammation, tissue growth or cell proliferation, tissue remodeling (e.g., maturation and cell differentiation), or any combination thereof.

In some embodiments, the method comprises treating a chronic wound. In some embodiments, a chronic wound comprises a wound that does not heal in the orderly set of phases, such as disclosed herein. In some embodiments, a chronic wound comprises a wound that does not heal or heals slower than as a wound would normally heals. In some embodiments, a substantial recovery of a wound of a healthy individual can be observed up until 3 months from the wound onset. In some embodiments, a chronic wound comprises a wound that does not heal, at least partially, within three months from the wound onset. In some embodiments, a chronic wound comprises a wound that does not heal, at least partially, within 0-0.5, 0.5-1, 1-2, 2-3, 3-4, 4-5, 5-6, 6-7, 7-8, 9-10, 10-11, or 11-12, months from the wound onset. Each possibility represents a separate embodiment of the present invention. In some embodiments, wound healing of a chronic wound comprises at least one prolonged healing phase, selected from: blood clotting, inflammation, tissue growth, tissue remodeling, or any combination thereof, as compared to a control subject. In some embodiments, a chronic wound comprises longer inflammatory stage, as compared to a control subject. In some embodiments, longer healing phase comprises increased healing phase of at least one phase selected from: blood clotting, inflammation, tissue growth, tissue remodeling, or any combination thereof, by at least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, or 1000%, as compared to a control subject. Each possibility represents a separate embodiment of the present invention. In some embodiments, the method reduces the time necessary to the wound to heal. In some embodiments, treating comprises accelerating wound healing, or shortening the duration or period of time of wound healing compared to a control subject. In some embodiments, the method disclosed herein accelerates the wound healing process. In some embodiments, shortening or accelerating is by at least 5%, 10%, 15%, 20%, 25%, 30%, 35%, 40%, 45%, 50%, 55%, 60%, 65%, 70%, 75%, 80%, 85%, 90%, 95% or 99%, compared to a wound healing in a control subject. Each possibility represents a separate embodiment of the present invention.

As used herein a “control subject” refers to a subject that lacks a disease or disorder that prevents or impairs a normal healing process of a wound, as disclosed herein. In some embodiments, a control subject comprises a healthy subject. In some embodiments, a control subject comprises a subject that is not afflicted with a metabolic disease or disorder, as disclosed herein. In some embodiments, a control subject is not afflicted with a metabolic disorder selected from: pre-diabetes, type 1 diabetes, type 2 diabetes, insulin resistance or insulin-resistance related, gestational diabetes, hyperglycemia, or any combination thereof. In some embodiments, a control subject refers to a subject in which a wound would heal, at least partially, until 3 months from the wound onset. In some embodiments, a control subject refers to a subject in which a wound would heal, at least partially, within 0-0.5, 0.5-1, 1-2, 2-3, 3-4, 4-5, 5-6, 6-7, 7-8, 9-10, 10-11, or 11-12, months from the wound onset.

In some embodiments, the method disclosed herein is to treat an acute wound.

As used herein, the term “an acute wound” refers to an injury to the skin that occurs suddenly rather than over time. In some embodiments, the method is for treating an acute deep wound, damaging internal tissues (e.g., blood vessels, nerves or muscles).

In some embodiments, the wound is accompanied with pain. In some embodiments, the subject is afflicted with pain. In some embodiments, the wound is accompanied with infection. In some embodiments, the subject is afflicted with infection. In some embodiments, the wound is caused by a traumatic trigger. (e.g., surgery or accidental trauma). In some embodiments, a chronic wound results from at least one pathology selected from: systemic infection, vascular pathology, immune pathology, nerve insufficiency, neoplasia, a metabolic disorder, and any combination thereof. In some embodiments, a chronic wound results from ischemia, reperfusion injury, bacterial colonization, or any combination thereof. Examples for chronic wounds are known in the art. Several examples include, but are not limited to, venous and arterial ulcers, diabetic ulcers, and pressure ulcers. In some embodiments, the method is for treating a diabetic wound or ulcers.

As used herein, a microRNA (miR) molecule is a small non-coding RNA molecule. In some embodiments, a miR molecule comprises a single-stranded nucleotide molecule. In some embodiments, a miR molecule comprises a double-stranded nucleotide molecule. In some embodiments, a miR molecule functions in RNA silencing and/or post-transcriptional regulation of gene expression. In some embodiments, a miR molecule comprises a precursor of miR molecule. In some embodiments, the miR precursor is cleaved to produce a mature miR molecule. In some embodiments, the mature miR is initially transcribed as a longer primary transcript, termed primary miR (pri-miR). In some embodiments, a pri-miR molecule comprises a miR precursor sequence (pre-miR) and a mature miR sequence (mmiR). In some embodiments, one of the two strands of the pri-miR molecule includes the mmiR. In some embodiments the pri-miR is cleaved to yield about 70 nucleotides precursor miR molecule (pre-miR), and 22 nucleotides mature miR molecule (mmiR). In some embodiments, the pre-miR is transported to the cytoplasm and further processed to produce a short, partially double-stranded RNA, in which one strand is the mmiR. In some embodiments, the miR molecule disclosed herein comprises any form of the miR molecule, comprising pri-miR, pre-miR or the mmiR molecule thereof.

In some embodiments, the miR molecule disclosed herein comprises a primary (pri-miR) or a precursor miR (pre-miR) molecule. In some embodiments, pri-miR or pre-miR molecule comprises an RNA hairpin molecule. In some embodiments, a pri-miR or pre-miR molecule comprises 60-120 nucleotides. In some embodiments, a miR molecule comprises a mature miR molecule (mmiR). In some embodiments, a miR molecule comprises 15-27 nucleotides. In some embodiments, mmiR molecule comprises about 22 nucleotides. In some embodiments, a polyA polymerase can add 1-3 nucleotides to the 3′ end of the mature transcript of a miR molecule. In some embodiments, a miR molecule comprises about 22 nucleotides. In some embodiments, a miR molecule comprises about 23-25 nucleotides.

Methods for gene silencing by miR molecules are known in the art. In some embodiments, a miR molecule functions via base-pairing with a complementary sequence within a mRNA molecule. In some embodiments, the complemented mRNA molecule, paired with the miR molecule, is silenced. Examples for gene silencing methods by miR molecules include, but are not limited to: (1) cleavage of a mRNA strand into two pieces, (2) destabilization of the mRNA through shortening of its poly (A) tail, (3) reduced translation efficiency of the mRNA into a protein by ribosome, and; (4) guidance argonaut (AGO) proteins to completely or partially complementary binding sites at the 3′ untranslated region (UTR) of target mRNA.

In some embodiments, the miR molecule disclosed herein belongs to miR-1 family. In some embodiments, the miR molecule comprises a miR precursor selected from miR-1 family. In some embodiments, the miR molecule comprises a mature miR product of miR-1 family, e.g., a mature molecule derived from cleavage of a member of miR-1 family.

As used herein, “miR-1 family refers” to miRBase family #MIPF0000038, comprising miR precursors, and the mature miR molecules thereof. In some embodiments, the miR molecule comprises a mammalian miR. In some embodiments, the miR molecule comprises a human miR. In some embodiments, the miR molecule comprises at least one molecule selected from: miR-1-1, miR-1-2, miR-1-3p, and any combination thereof. In some embodiments, miR-1-3p comprises a mature product of miR-1-1, miR-1-2, and any combination thereof. In some embodiments, the miR molecule comprises at least one molecule selected from: Homo sapiens miR-1-1 (hsa-miR-1-1), Homo sapiens miR-1-2 (hsa-miR-1-2), Homo sapiens miR-1-3p (hsa-miR-1-3p), and any combination thereof. In some embodiments, the miR molecule comprises hsa-miR-1-3p.

In some embodiments, the method comprises administering to a subject in need thereof a therapeutically effective amount of a pharmaceutical composition comprising an agent capable of increasing expression, abundance, or both, of a miR molecule belonging to miR-1 family, or a mature miR product thereof. In some embodiments, the agent is capable of increasing transcription of a gene, resulting in increased abundance of the miR molecule disclosed herein. In some embodiments, the agent is capable in increasing the miR abundance. In some embodiments, the agent increases a miR molecule expression by at least 1-1.5-, 1.6-2.0-, 2.1-3.0-, 3.1-4.0-, or 4.1-5.0-fold, 5.1-10-, or 10.1-100-fold. Each possibility represents a separate embodiment of the present invention. In some embodiments, the agent increases a miR molecule expression by at least 10%, 20%, 30%, 40%, 50%, 60%, 70%, 80%, 90% or 99%. Each possibility represents a separate embodiment of the present invention. In some embodiments, the agent increases a miR molecule expression by 10-20%, 21-30%, 31-40%, 41-50%, 51-60%, 61-70%, 71-80%, 81-90%, or 91-100%. Each possibility represents a separate embodiment of the present invention.

As used herein, “an agent” refers to any molecule, comprising a small molecule, a polynucleotide, or a polypeptide, that is able to silence or inhibit the target gene (either direct or downstream) of the miR molecule disclosed herein. Examples for such an agent include an antibody or fragment thereof, a peptide, a small molecule, an RNA molecule, or a DNA molecule, as long as the agent inhibits the target gene, either a direct gene target or in a downstream signaling pathway of the direct gene target, identical to that of the miR molecule disclosed herein.

In some embodiments, the agent comprises at least one of: pri-miR, pre-miR, and mature miR thereof, from the miR-1 family. In some embodiments, miR expression can be controlled by transcription factors or possibly by other miR molecules. In some embodiments, the agent comprises a molecule that inhibits a regulatory miR molecule of at least one of: hsa-miR-1-3p, hsa-miR-1-1 and hsa-miR-1-2.

In some embodiments, the agent comprises a miR molecule mimics.

As used herein, a “miR molecule mimics” refers to a nonnatural or an artificial RNA fragment, that its silencing target within the mRNA molecule is identical to the silencing target of the homologous endogenous miR molecule. In some embodiments, a miR mimics comprises a double stranded RNA molecule. In some embodiments, a miR mimics comprises a single stranded RNA molecule. In some embodiments, a miR mimics comprises 15-30 nucleotides. In some embodiments, a miR mimics comprises at least one of: 15-30, 17-28, 19-26, 20-25, 21-24, or 21-23 nucleotides. Each possibility represents a separate embodiment of the present invention. In some embodiments, the agent is capable in silencing the miR target gene. In some embodiments, the agent is capable in silencing at least one target gene of a miR molecule from miR-1 family or their mature products thereof. In some embodiments, the agent comprises a miR mimics of hsa-miR-1-3p, or its precursor thereof. In some embodiments, the agent comprises a miR mimics of at least one of hsa-miR1-3p, hsa-miR-1-1, hsa-miR-1-2, and any combination thereof.

In some embodiments, the agent is capable in silencing a target gene of miR-1 family, its precursor thereof, or its mature product thereof. In some embodiments, the agent is capable in silencing at least one molecule selected from: hsa-miR-1-1, hsa-miR-1-2, hsa-miR-1-3p, and any combination thereof. In some embodiments, the agent is capable in silencing a target gene of hsa-miR-1-3p. In some embodiments, the target gene comprises a direct target gene of the miR molecule selected from miR-1 family and their mature products thereof. In some embodiments, the target gene comprises a gene that is silenced by at least one of: hsa-miR-1-1, hsa-miR-1-2 and hsa-miR-1-3p. In some embodiments, the target gene comprises a direct target gene that is silenced by hsa-miR-1-3p. In some embodiments, the target gene comprises a direct target gene that its translation is inhibited due to the miR binding to the mRNA transcribed by the gene. In some embodiments, the target gene comprises a gene in the downstream pathway of the gene that is silenced by the miR molecule from the miR-1 family. In some embodiments, the target gene comprises a downstream gene, i.e. in the downstream pathway of the genes that are activated or inhibited by at least one of: hsa-miR-1-1, hsa-miR-1-2 and hsa-miR-1-3p. In some embodiments, the target gene comprises a gene in the downstream pathway of the gene that is silenced by hsa-miR-1-3p. In some embodiments, the target gene comprises a downstream gene that is activated or inhibited by the endogenous hsa-miR-1-3p.

In some embodiments, the agent is capable in silencing protein tyrosine kinase 9 (PTK9). In some embodiments, PTK9 is a target gene of the agent disclosed herein. PTK9, also known as twinfilin actin binding protein 1 (TWF1), UniProtKB #: Q12792. TWF1 comprises an actin-binding protein involved in motile and/or morphological processes. In some embodiments, the agent is capable in silencing phosphatidylinositol-4-phosphate 3-kinase C2 domain-containing alpha polypeptide (PIK3C2A). In some embodiments, PIK3C2A is a target gene of the agent disclosed herein. PIK3C2A, UniProtKB #: 000443, belongs to the phosphoinositide 3-kinase (PI3K) family. In some embodiments, PIK3C2A plays a role in a signaling pathway involved in at least one of: cell proliferation, oncogenic transformation, cell survival, cell migration, and intracellular protein trafficking.

In some embodiments, silencing comprises reduced transcription of the miR target gene to mRNA. In some embodiments silencing comprises reduced translation of the mRNA transcribed by the target gene to a protein. In some embodiments, silencing comprises induced destabilization or degradation of a transcribed mRNA, or a protein translated therefrom, by the miR target gene. In some embodiments, silencing is by at least 15%, 20%, 25%, 30%, 35%, 40%, 45%, 50%, 55%, 60%, 65%, 70%, 75%, 80%, 85%, 90%, 95% or 99%. Each possibility represents a separate embodiment of the present invention. In some embodiments, the agent is capable in silencing the miR target gene by one of: 10-20%, 21-30%, 31-40%, 41-50%, 51-60%, 61-70%, 71-80%, 81-90%, or 91-100%. Each possibility represents a separate embodiment of the present invention.

In some embodiments, the agent comprises the miR molecule. In some embodiments, the agent comprises a precursor or a primary form of the miR molecule. In some embodiments, the agent disclosed herein is within a pharmaceutical composition. In some embodiments, the pharmaceutical composition comprises a liposome. As used herein, a liposome is a spherical-shaped vesicle that is composed of one or more phospholipid bilayers. In some embodiments, the pharmaceutical composition comprises a carrier. In some embodiments, the pharmaceutical composition comprises a carrier and a liposome. In some embodiments, the agent disclosed herein is encapsulated within the liposome. In some embodiments, at least one of the miR molecule, a precursor thereof, a mature product thereof, and a combination thereof, is encapsulated within the liposome. In some embodiments, at least one of the miR molecule, a precursor thereof, a mature product thereof, and a combination thereof, is bound to a carrier. In some embodiments, at least one of the miR molecule, a precursor thereof, a mature product thereof, and a combination thereof, is encapsulated within a liposome and bound to a carrier. In some embodiments, the carrier comprises a cationic polysaccharide. In some embodiments, the carrier comprises chitosan.

In some embodiments, there is provided a method for treating a wound in a subject in a need thereof, the method comprising: (a) providing a composition comprising a fibroblast being characterized by increased expression, abundance, or both, of a miR molecule from miR-1 family; and, (b) administering a therapeutically effective amount of the composition from step (a) to the subject, thereby treating a wound in the subject.

In some embodiments, the method further comprises a step preceding step (a), comprising obtaining a fibroblast and contacting the fibroblast with an agent capable of increasing expression, abundance, or both, of a miR molecule belonging to miR-1 family, in the fibroblast.

In some embodiments, increasing expression, abundance, or both, of a miR molecule belonging to miR-1 family, in the fibroblast, is increasing by at least one of: 1-1.5-, 1.6-2.0-, 2.1-3.0-, 3.1-4.0-, or 4.1-5.0-, 5.1-10-, or 10.1-100-fold. Each possibility represents a separate embodiment of the present invention. In some embodiments, the increased miR molecule expression in fibroblast is by at least 10%, 20%, 30%, 40%, 50%, 60%, 70%, 80%, 90% or 99%. Each possibility represents a separate embodiment of the present invention. In some embodiments, the increased miR molecule expression in a fibroblast is by 10-20%, 21-30%, 31-40%, 41-50%, 51-60%, 61-70%, 71-80%, 81-90%, or 91-100%. Each possibility represents a separate embodiment of the present invention. In some embodiments, the increased expression, abundance, or both, of a miR molecule belonging to miR-1 family, in the fibroblast, is compared to a control fibroblast. As used herein, “a control fibroblast” refers to a fibroblast from a wound in a subject in need thereof, that is not transfected or modified to express the agent and/or the miR molecule disclosed herein. In some embodiments, a control fibroblast is a non-modified fibroblast from a chronic wound.

In some embodiments, increased expression, abundance, or both of a miR molecule belonging to miR-1 family leads to similar expression, abundance, or both of the miR molecule, as compared to at least one of: (a) a fibroblast obtained from a healthy individual, (b) a fibroblast obtained from an individual that lacks a pathology that leads to a chronic wound or impaired wound healing process; or, (c) a fibroblast obtained from the same subject in need thereof, derived from a healthy tissue, lacking a chronic wound pathology.

Various methods for expression of a miR molecule in a cell are known in the art. Examples are described in Fan J et al. “A simplified system for the effective expression and delivery of functional mature microRNAs in mammalian cells.” Cancer Gene Ther. 2020; 27:424-437, herein incorporated in its entirety. In some embodiments, a fibroblast can be transfected by the miR molecule, the miR mimics, its precursor (pre-miR) or its precursor or primary form (pri-miR) thereof. In some embodiments a transfection reagent can be used for efficient transfection. In some embodiments, expression of a miR molecule can be accomplished by the use of an expression vector. In some embodiments, expression comprises overexpression or upregulation. In some embodiments expression of a miR molecule is performed by an expression vector typically used for expression of at least one of: miR mimics, short hairpin

RNA-like (shRNA-like) or intronic miR (e.g., intron-derived miR that is derived from the processing of gene introns). In some embodiments at least one of: pri-miR, pre-miR, and mature miR is expressed in the fibroblast disclosed herein. In some embodiments, expression of a miR mimics in a cell is accomplished by delivery of a plasmid or through a viral or a bacterial vector. In some embodiments, the agent or the miR molecule is forward transfected in a cell (e.g., the transfected reagent and the agent, the miR molecule, or a combination thereof, are added to a cultured cell). In some embodiments, the agent or the miR molecule is reverse transfected in a cell (e.g., the cells are not cultured prior to the addition of reagent transfection and addition of the agent, the miR molecule, or a combination thereof).

In some embodiments, the method disclosed herein increases a fibroblast proliferation. In some embodiments, the method increases a fibroblast migration. In some embodiments, administration to a subject in need thereof one of: (a) an agent that induces expression of a miR molecule from miR-1 family, (b) a miR molecule from miR-1 family, precursor thereof or mature product thereof, (c) a fibroblast expressing the agent or the miR molecule from miR-1 family; and, (d) any combination thereof, leads to increased proliferation and/or migration of a fibroblast. In some embodiments, the fibroblast that is transfected to express the miR molecule, or the agent disclosed herein, comprises enhanced proliferation and/or migration ability. In some embodiments, in vitro expression of the agent or the miR molecule leads to increased proliferation and/or migration ability. In some embodiments, a fibroblast within a subject's wound, or the wound niche, absorbs the administered agent or the miR molecule, and displays increased proliferation and/or migration ability. In some embodiments, a modified fibroblast, expressing elevated levels of the agent or the miR molecule disclosed herein, is transplanted into the wound and secretes a growth factor, or a cytokine, that induces the proliferation and or migration of an endogenous non modified fibroblast within the wound. In some embodiment the growth factor comprises vascular endothelial growth factor (VEGF). In some embodiments, the treatment leads to increased proliferation and/or migration of an endothelial cell. In some embodiments, increased proliferation and/or migration of at least one of: a fibroblast, an endothelial cell, and any combination thereof is observed as early as 6 hours post treatment administration. In some embodiments, increased proliferation comprises at least 10%, at least 20%, at least 30%, at least 40%, at least 50%, at least 60%, at least 70% at least 80%, at least 90%, or at least 95% increase in fibroblast proliferation. In some embodiments, increased migration capacity comprises at least 10%, at least 20%, at least 30%, at least 40%, at least 50%, at least 60%, at least 70% at least 80%, at least 90%, or at least 95% increase in fibroblast and/or endothelial cell migration. In some embodiments, the method disclosed herein increases the closure of a wound in a subject in need thereof. In some embodiments increased closure is by at least 1.1-1.2, 1.3-1.4, 1.5-1.6, 1.7-1.8, 1.9-2.0, or 2.1-3.0-fold, compared to a control wound. As used herein, a “control wound” refers to a similar wound in shape and/or damage severity, from similar pathology, that is not treated by the method disclosed herein. In some embodiments a “control wound” refers to an untreated chronic wound. In some embodiments, a “control wound” refers to a chronic wound that is not treated by the method or the pharmaceutical composition of the invention. In some embodiments, a control wound comprises a diabetic wound that is not treated by the method and/or the composition disclosed herein.

In some embodiments, the method disclosed herein increases vascular endothelial growth factor (VEGF) expression in a fibroblast. In some embodiments, increased VEGF expression comprises increased mRNA expression. In some embodiments, increased VEGF expression comprises increased protein expression. In some embodiments, increased VEGF expression comprises increased VEGF protein secretion. In some embodiments, increased VEGF expression in a fibroblast is by at least 1.1-1.2, 1.3-1.4, 1.5-1.6, 1.7-1.8, 1.9-2.0, or 2.1-3.0-fold, compared to a control fibroblast.

In some embodiments, the fibroblast is obtained or derived from the same subject in need thereof, having the wound. In some embodiments, the treatment comprises an autologous transplantation of the fibroblast. In some embodiments, the fibroblast is obtained or derived from a different subject than the subject in need thereof. In some embodiments, the fibroblast is obtained from a healthy individual. In some embodiments, the fibroblast donor lacks a pathology leading to a chronic wound. In some embodiments, the method comprises an allogeneic transplantation of the fibroblast.

In some embodiments, the subject in need thereof is afflicted with a disease or disorder that prevents a normal healing process of a wound. In some embodiments the subject is afflicted with a disease or disorder that is known to be accompanied with a chronic wound, as disclosed herein. In some embodiments, the subject is afflicted with a disease that prolongs the healing time of a wound. In some embodiments, the subject is afflicted with a disease or disorder selected from: a metabolic disease or disorder, anemia, arterial circulatory disorder, gout, chronic venous insufficiency and an autoimmune disease. In some embodiments, the autoimmune disease comprises rheumatoid arthritis (RA), lupus or scleroderma. In some embodiments the subject is afflicted with a metabolic disease or disorder. In some embodiments, a metabolic disease or disorder is selected from: pre-diabetes, type 1 diabetes, type 2 diabetes, insulin resistance or insulin-resistance related, gestational diabetes, hyperglycemia, obesity, diabetic dyslipidemia, hyperlipidemia, hypertriglyceridemia, hyper-fattyacidemia, hypercholesterolemia, and hyperinsulinemia. In some embodiments, the subject is afflicted with a metabolic disorder selected from: pre-diabetes, type 1 diabetes, type 2 diabetes, insulin resistance or insulin-resistance related, gestational diabetes, and hyperglycemia.

In some embodiments, the miR molecule belonging to miR-1 family comprises a nucleic acid sequence consisting of: TGGAATGTAAAGAAGTATGTAT (SEQ ID NO: 1). In some embodiments, any one of the agent or the miR molecule disclosed herein comprises a functional analog thereof having at least 80% sequence homology to SEQ ID NO: 1.

In some embodiments, the miR molecule belonging to miR-1 family comprises a nucleic acid sequence consisting of: TGGGAAACATACTTCTTTATATGCCCATATGGACCTGCTAAGCTATGGAATGTAAA GAAGTATGTATCTCA (SEQ ID NO: 2). In some embodiments, any one of the agent or the miR molecule disclosed herein comprises a functional analog thereof having at least 80% sequence homology to SEQ ID NO: 2.

In some embodiments, the miR molecule belonging to miR-1 family comprises a nucleic acid sequence consisting of: ACCTACTCAGAGTACATACTTCTTTATGTACCCATATGAACATACAATGCTATGGA ATGTAAAGAAGTATGTATTTTTGGTAGGC (SEQ ID NO: 3). In some embodiments, any one of the agent or the miR molecule disclosed herein comprises a functional analog thereof having at least 80% sequence homology to SEQ ID NO: 3.

In some embodiments, hsa-miR-1-3p comprises SEQ ID NO: 1. In some embodiments, hsa-miR-1-1 comprises SEQ ID NO: 2. In some embodiments, hsa-miR-1-2 comprises SEQ ID NO: 2.

In some embodiments, any one of: the agent or the miR molecule disclosed herein, comprises a functional analog of any one of SEC ID NOs: 1-3. In some embodiments, any one of: the agent or the miR molecule disclosed herein comprises a functional analog, having at least 80%, at least 85%, at least 90%, at least 95%, or at least 97%, sequence homology to any one of SEQ ID NOs: 1-3. In some embodiments, any one of: the agent or the miR molecule disclosed herein comprises a functional analog, having one of: 80-99%, 85-99%, 90-99%, 95-99%, or 97-99% sequence homology to any one of SEQ ID NOs: 1-3.

The term “analog” as used herein, refers to a nucleotide molecule that is similar, but not identical, to the miR molecule of the invention that still is capable in improving or accelerating a wound healing process. An analog may have deletions, additions or mutations that result in nucleotide sequence that is different than the nucleotide sequence of the miR molecule of the invention. It should be understood that all analogs of the miR molecule of the invention would still be capable of improving or accelerating wound healing process. Further, an analog may be analogous to a fragment of the nucleotide of the invention, however, in such a case the fragment must comprise at least 15 consecutive nucleotides of the miR molecule of the invention. An analog may be a single-strand nucleotide sequence. An analog may be a double-strand nucleotide sequence.

In some embodiments, the agent or the miR molecule comprises the nucleic acid sequence set forth in SEQ ID NO: 1. In some embodiments, the agent or the miR molecule comprises a functional analog having at least at least 80%, at least 85%, at least 90%, at least 95%, or at least 97%, sequence homology to SEQ ID NO: 1. In some embodiments, the agent or the miR molecule comprises a functional analog, having one of: 80-99%, 85-99%, 90-99%, 95-99%, or 97-99% sequence homology to SEQ ID NO: 1.

In some embodiments, the method disclosed herein comprises local administration of any one of: the agent, the miR molecule, or the fibroblast comprising the agent or the miR molecule, directly to the wound of the subject in need thereof. As used herein, the term “local administration” refers to a direct delivery of the agent, the miR molecule, or the fibroblast comprising the agent, into the exposed tissue of an open skin wound.

### Pharmaceutical Compositions

According to some embodiments there is provided a pharmaceutical composition comprising a nucleic acid molecule being a miR molecule belonging to miR-1 family, for use in the treatment of a wound in a subject in need thereof.

According to some embodiments there is provided a pharmaceutical composition comprising a nucleic acid molecule being a miR molecule belonging to miR-1 family, for use in the treatment of a wound in a subject in need thereof. In some embodiments, the pharmaceutical composition comprises an agent that enhances the expression, abundance, or both of at least one of: hsa-miR-1-1, hsa-miR-1-2, hsa-miR-1-3p, and any combination thereof. In some embodiments the pharmaceutical composition comprises an agent that silences or inhibits the target gene of at least one of: hsa-miR-1-1, hsa-miR-1-2, hsa-miR-1-3p. In some embodiments, the agent inhibits the transcription of a target gene to mRNA, either direct or in a downstream pathway, of hsa-miR-1-3p. In some embodiments, the agent inhibits the translation of the mRNA to a protein, of a target gene, either direct or in a downstream pathway, of hsa-miR-1-3p. In some embodiments, the agent increases at least one of: destabilization or degradation, of a mRNA transcribed, or a protein translated from a target gene, either direct or in the downstream pathway, of hsa-miR-1-3p. In some embodiments, silencing is by at least 15%, 20%, 25%, 30%, 35%, 40%, 45%, 50%, 55%, 60%, 65%, 70%, 75%, 80%, 85%, 90%, 95% or 99%. Each possibility represents a separate embodiment of the present invention. In some embodiments, the agent is capable in silencing the miR target gene by one of: 10-20%, 21-30%, 31-40%, 41-50%, 51-60%, 61-70%, 71-80%, 81-90%, or 91-100%. Each possibility represents a separate embodiment of the present invention.

In some embodiments the miR molecule belonging to miR-1 family comprises a mature miR molecule, a precursor thereof, or a combination thereof.

In some embodiments, the pharmaceutical composition comprises a miR molecule selected from: SEQ ID NO: 1 or a functional analog thereof having at least 80% sequence homology thereto, SEQ ID NO: 2 or a functional analog thereof having at least 80% sequence homology thereto, SEQ ID NO: 3 or a functional analog thereof having at least 80% sequence homology thereto, or any combination thereof.

In some embodiments, the mature miR molecule comprises the nucleic acid sequence set forth in SEQ ID NO: 1. In some embodiments, the pharmaceutical composition further comprises any one of: a liposome, a carrier, and both. In some embodiments, any one of: mature miR molecule, precursor thereof, or a combination thereof, is encapsulated within the liposome. In some embodiments, any one of: miR molecule, precursor thereof, or a combination thereof, is bound to a carrier. In some embodiments, the carrier comprises a cationic polysaccharide. In some embodiments, the carrier comprises chitosan.

In some embodiments, there is a pharmaceutical composition comprising a modified fibroblast being characterized by increased expression, abundance, or both, of a miR molecule belonging to miR-1 family, compared to a control fibroblast.

In some embodiments, the modified fibroblast within the pharmaceutical composition is obtained by contacting a fibroblast derived or obtained from a diabetic subject with an agent capable of increasing expression, abundance, or both, of a miR molecule belonging to miR-1 family.

In some embodiments the pharmaceutical composition comprises an agent, selected from the group consisting of: miR molecule, a precursor thereof, and a combination thereof.

In some embodiments, the pharmaceutical composition is for use in the treatment of a wound. In some embodiments, the wound is a wound of or in a subject. In some embodiments, a subject in need thereof is a subject comprising, characterized by, having a wound, or any combination thereof. In some embodiments, the pharmaceutical composition is locally administered. In some embodiments, local administration comprises administration directly to the wound of the subject. In some embodiments, local administering comprises at least one of: intradermal (ID) injection, subcutaneous (SC) injection and intramuscular (IM) injection. In some embodiments, the route of administration depends upon the wound severity. In some embodiments, a deep wound, damaging an internal layer, or ulcer, requires administering the pharmaceutical composition directly to the inner injured layer. In some embodiments, the pharmaceutical composition is injected SC (e.g., delivered the fat layer). In some embodiments, the pharmaceutical composition is injected IM (e.g., delivered to the muscle). In some embodiments, the pharmaceutical composition is injected ID (e.g., delivered underneath the skin). In some embodiments, the pharmaceutical composition is formulated for local administration. In some embodiments, the pharmaceutical composition is formed as one of: a suspension, a spray, a cream, an ointment, or any combination thereof.

In some embodiments, the pharmaceutical composition is systemically administered. In some embodiments, the severity of the wound requires a systemic administration of the pharmaceutical composition (e.g., intravenous administration). In some embodiments, the treatment with the pharmaceutical composition of the invention is combined with antibiotics. In some embodiments, a synergistic effect is achieved by combination of the pharmaceutical composition disclosed herein and antibiotics therapy. In some embodiments, combined treatment of the pharmaceutical composition with antibiotics is required for an infected wound or ulcer. Examples for an antibiotic regimen required for a mild soft tissue infection include oral antibiotics (e.g., dicloxacillin, cephalexin, or clindamycin). In some embodiments, severe soft tissue infection is further treated intravenously with one of: ciprofloxacin plus clindamycin; piperacillin/tazobactam; or imipenem/cilastatin. In some embodiments, the antibiotics regimen depends upon the bacterium/bacteria strains identified, either in the wound or ulcer, or in the blood. In some embodiments, the wound treatment further requires a culture-guided definitive therapy.

The term “therapeutically effective amount” refers to the concentration of one of: (a) the agent or miR molecule of the invention; and (b) the fibroblast of the invention, that is normalized to body weight (BW) and is effective to treat a disease or disorder in a mammal. The term “a therapeutically effective amount” refers to an amount effective, at dosages and for periods of time necessary, to achieve the desired therapeutic or prophylactic result. A physician of ordinary skill can readily determine and prescribe the effective amount of the bioactive agent required. The exact dosage form and regimen would be determined by the physician according to the patient's condition. The dosage administered will be dependent upon the age, health, and weight of the recipient, kind of concurrent treatment, if any, frequency of treatment, and the nature of the effect desired.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal. The dosage administered will be dependent upon the age, health, and weight of the recipient, kind of concurrent treatment, if any, frequency of treatment, and the nature of the effect desired.

In some embodiments, the route of administration of the pharmaceutical composition disclosed herein comprises an intravenous route, an intramuscular route, a subcutaneous route, or an oral delivery route. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate compositions it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. In some embodiments, the composition of the invention comprising oral delivery. In some embodiments, the composition of the invention comprises an oral composition. In some embodiments, the composition of the invention further comprises orally acceptable carrier, excipient, or a diluent.

As used herein, the terms “subject” or “individual” or “animal” or “patient” or “mammal” refers to any subject, particularly a mammalian subject, for whom therapy is desired, for example, a human.

In some embodiments, the pharmaceutical composition comprises a pharmaceutically acceptable carrier, adjuvant, or excipient.

As used herein, the term “carrier”, “adjuvant”, or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the term “about” when combined with a value refers to plus and minus 10% of the reference value. For example, a length of about 1000 nanometers (nm) refers to a length of 1000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Participants

The participants are patients from the Department of Internal Medicine D and/or Institute of Endocrinology and Diabetes in Rambam Health Care Campus. Dermal biopsies were taken by surgeon at plastic surgery clinic in the same campus. The biopsies were taken under sterile conditions. Each donor provided consent after a full explanation of the research.

### Fibroblasts Extraction

Biopsies were transferred to biosafety cabinet. After washing the biopsies with ethanol 70%, epidermis and cutaneous layers were removed. Then, dermis layers were dissected into pieces, were transferred into tissue culture plates/dishes and were covered with BIO-AMF complete medium (Bio-ind. 01-194-1B). Once fibroblasts appeared in the dish (7-14 days), the medium was changed to DMEM (Bio-ind. 01-055-1, 01-055-1A), supplemented with 10% heat-inactivated fetal bovine serum (FBS), over a period of 4 weeks in a dish/plate, with media supplementation every other day. Subsequently, as fibroblasts emerged from the primary explant, a brief trypsinization (0.25% trypsin) was used to separate and further expand the cells in 75 cm flasks. In all experiments, fibroblasts were used between passages 2 and 5.

### RNA Isolation and Quantification

RNA was isolated using the mir Vana™ miRNA Isolation Kit (Ambion, Austin, TX, USA) according to the manufacturer's instructions. The purity and quantity of RNA were assessed using the NanoDrop ND-1000 spectrophotometer (Thermo Scientific, Wilmington, DE, USA). The samples were used immediately or stored at −80° C. for future use.

### RNA Quality Control

Quality control examination of total RNA was performed by examination of the peak of small fragments using TapeStation 4200 (Agilent). The RINe value of all samples was in the range of 9.7-10.

### MiRNA-Seq, Library Construction and Sequencing

MiRNAseq analysis was performed in the Genomics Center of the Biomedical Core Facility, Faculty of Medicine, Technion. For differential expressed miRNAs, human fibroblasts from diabetic donors and healthy donors were sequenced. RNA was isolated from six donors of each group. The samples were diluted to a final concentration of 10 ng/μl. Twelve miRNAseq libraries were produced according to manufacture protocol (QIAseq miRNA library kit, cat no. 331502) using 150 ng total RNA. All libraries were mixed into a single tube with equal molarity.

The miRNAseq data was generated on Illumina NextSeq500 with the recommended parameters of 75 single read, high output mode (Illumina, cat no. 20024906).

### Next Generation Sequencing Quality Control, Alignment and Counting

Primary analysis was done online, using QIAGEN's GeneGlobe Data Analysis Center (www.qiagen.com/data analysis), resulting with miRNA counts.

### Descriptive Analysis

A statistical analysis was preformed using DESeq2 R package (version 1.30.0) (Genome Biology 2014 15:550). The similarity between samples was evaluated within DESeq2 package using correlation matrix, shown in principal component analysis (PCA). The latter is drawn from the 500 most variable genes.

### Differential Expression Analysis

Differentially expressed (DE) miRNAs were identified using statistical analysis from the above step. The fold change in miRNA expression between two groups was considered significant if FDR-p<0.05 and fold change <−2 or >2 fold.

Real Time PCR Validation of DE miRNAs

The RNA samples from healthy and diabetic donors that were used for sequencing were also used to validate RNA-Seq differential expression. cDNA was synthesized using TaqMan® MicroRNA Reverse Transcription Kit (Applied Biosystems, Foster City, CA, USA) and miRNA was quantified in QuantStudio 3 using TaqMan™ Fast Advanced Master Mix (Applied Biosystems). A non-coding RNA U6 was used as an endogenous control for normalization, and fold change was calculated by ΔΔCt method.

### Transfection

MiRNA mimic, alexa fluor red positive control and negative control #1 mimics were purchased from Invitrogen and Ambion (Thermo Fisher Scientific), respectively. The fibroblasts were plated and transfected 24 h later at 30-50% confluency, using miRNA-1-p3 (50 nM), mimic miR negative control (NC; 50 nM) or positive control (PC; 50 nM) mimics and Lipofectamine RNAiMAX reagent (Invitrogen). Transfection efficiency was evaluated by RT-PCR quantification of PTK9, a target gene of hsa-miR-1-3p.

### Cell Migration Assay

Cultured fibroblasts (2.5×10⁴ cells/well) in 96 wells plate were transfected with 50 nM miR-1-p3 or NC mimic and starved by DMEM supplemented with 2% FBS for 8 hours before starting the experiment. Cultured fibroblasts were wounded by scratching the cell monolayer in a straight line by a mechanical scratcher to create a scratch, followed by a wash using phosphate buffered saline (PBS) (Bio-ind 02-023-1A) and incubation for 24 hours. Phase images of wounded areas were taken every 1 hour after incubation, and cell migration was determined using IncuCyte ZOOM System.

### Western Blot

Cells were lysed with RIPA reagent; Total protein levels were determined using Bradford protein assay. Thereafter, 18 μg protein were separated through SDS-PAGE (Bio-Rad 002052232300) and electro-blotted onto nitrocellulose membranes (Thermo Fisher Scientific TS-88518). The membranes were blocked with bovine serum albumin (BSA), probed with primary antibodies against VEGF (1:1000) (Sigma-Aldrich), FGF-2 (1:200), N-cadherin (1:200) and GAPDH (1:500) (Santa Cruz) and then probed with the secondary antibody (Cell signaling). Protein bands were analyzed using Image J software (NIH, USA) and normalized to GAPDH.

### VEGF Levels

MiRNA transfection was performed using the Lipofectamine RNAiMAX reagent for primary cells (Invitrogen) in 60% to 70% confluent fibroblasts. To evaluate the effect of miRNA on VEGF production, cells were washed twice with PBS and starved overnight with DMEM containing 1% BSA. Insulin (100 nM) or fresh DMEM 1% BSA was added for an additional 24 hours. After incubation, media were collected for VEGF measurement by ELISA (R&D Systems Quantikine kit DVE00), and the cell lysate was collected for the measurement of protein concentration.

### Flow Cytometry

For characterization of fibroblasts phenotype, flow cytometry was performed. The antibodies are FITC-conjugated anti-CD90 (Becton-Dickinson Biosciences).

### Wound Model in Mice

For in vivo wound-healing experiments, a wound was created in 8-week-old male nude mice (nu/nu, 002019), obtained from the Jackson Laboratory. On the day of the surgery (day 0), mice were anesthetized, and the dorsal skin was marked using a standardized 1.0-cm² template. A full-thickness wound on the dorsal area was created by excising a 1-cm² section of skin (consisting of the epidermis, dermis, and underlying Panniculus carnosus). Wound closure was monitored and documented on days 0, 2, 4, 6, and 8.

### Example 1

### Donors Characteristics

The study was approved by the Institutional Review Board at Rambam health care campus, Haifa (IRB-067-17). The participants were patients at Department of Internal Medicine D and/or Institute of Endocrinology and Diabetes in Rambam Health Care Campus. Fibroblasts were obtained from 6 non-diabetic controls and 6 Type II diabetes (T2DM) patients. 9 biopsies were extracted from 9 diabetic donors but only 6 had derived fibroblasts. Demographical and clinical characteristics of the donors are presented in Table 1. As may be seen, the mean age of the control donors is 74±8.11 yrs and of the diabetic donors is 54±10.6. For the control donors, 5 (83.33%) of 6 are males, for the diabetic donors; 6 (100%) are males. The mean duration of diabetes is 16.6±6.1 yrs and the mean glycated hemoglobin (HbA1c) levels were 8.36%±0.43%. Body mass index (BMI) of the control group is 26.1±2.9 (kg/m2) versus 24±8.7 (kg/m2) for the diabetic group. Of the diabetic group, 1 (16.67%) has neuropathy, 2 (33.33%) suffer from hypertension (HTN), 1 (16.67%) has skin ulcer and 1 donor (16.67%) had an amputation.

TABLE 1
Baseline characteristics of donors

| | Control | T2DM | |
|---|---|---|---|
| n (%) | 6 (50%) | 6 | (50%) |
| Age, yrs (range) | 74 ± 8.11 | 54 ± 10.6 | |
| Male Sex, (% of n) | 5 (83.3%) | 6 | (100%) |
| Age at diagnosis, yrs (range) | | 58.2 ± 7.6 | |
| Duration, yrs (range) | | 16.6 ± 6.1 | |
| HbA1c (%) | | 8.36 ± 0.43 | |
| BMI (kg/m2) | 26.1 ± 2.9 | 24.5 ± 8.7 | |
| Neuropathy, n (%) | 0 | 1 | (16.67%) |
| HTN, n (%) | 0 | 2 | (33.33%) |
| Skin ulcer, n (%) | 0 | 1 | (16.67%) |
| Any amputation, n (%) | 0 | 1 | (16.67%) |

### Example 2

### Fibroblasts Characterization

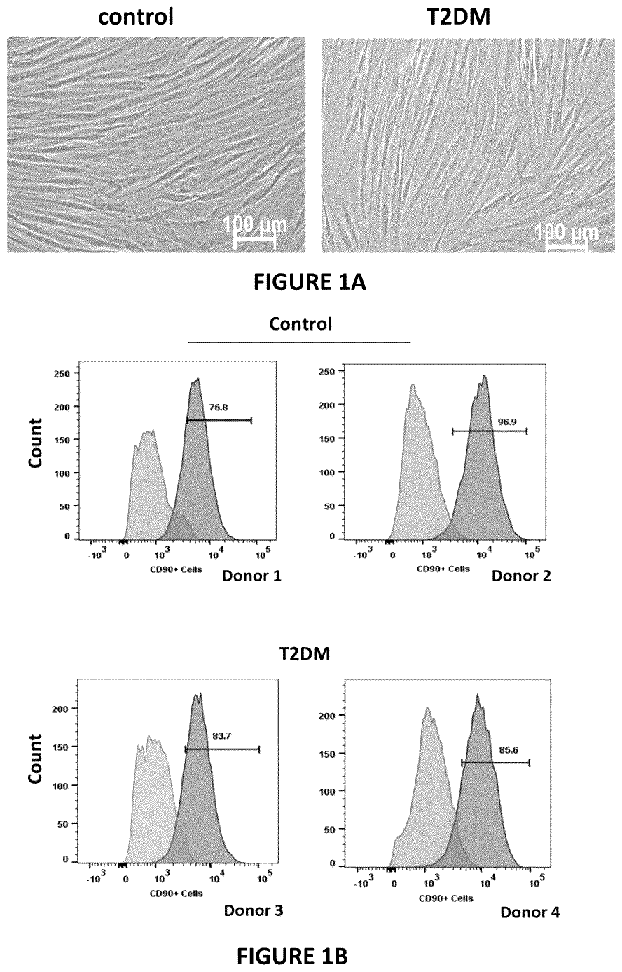

Biopsies were extracted in sterile conditions and immediately dissected in biosafety cabinet. Epidermis and cutaneous layers were removed, and the dermis layers were dissected into pieces and were transferred to tissue culture plates. Fibroblasts released from biopsies 4-6 weeks post dissection are presented in FIG. 1A.

The released cells were validated as fibroblasts by flow cytometry. FITC-conjugated anti-CD90 antibody is considered as a defining fibroblastic marker. The inventors confirmed that at least 75% of the released cells are fibroblasts, given the CD90+ cell count (FIG. 1B).

### Example 3

### Differentially Expressed miRNAs in Diabetic Fibroblasts

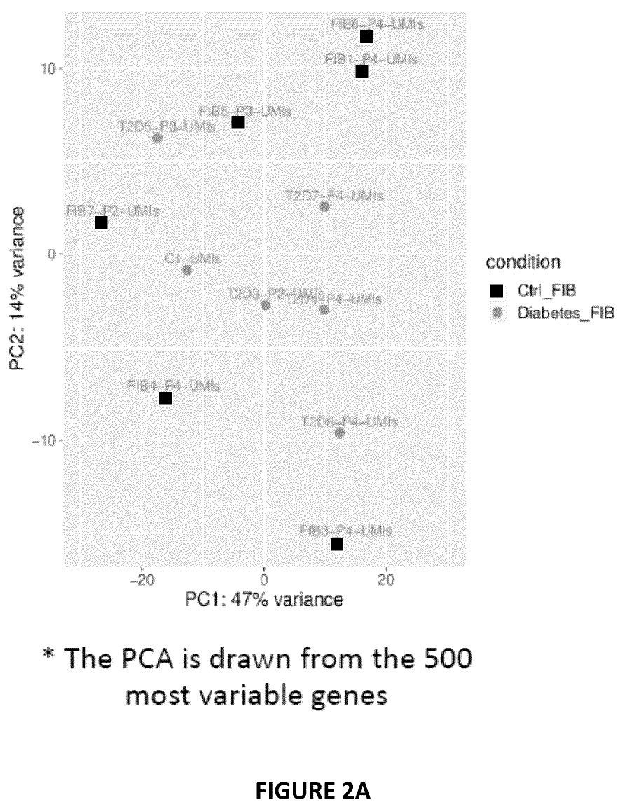

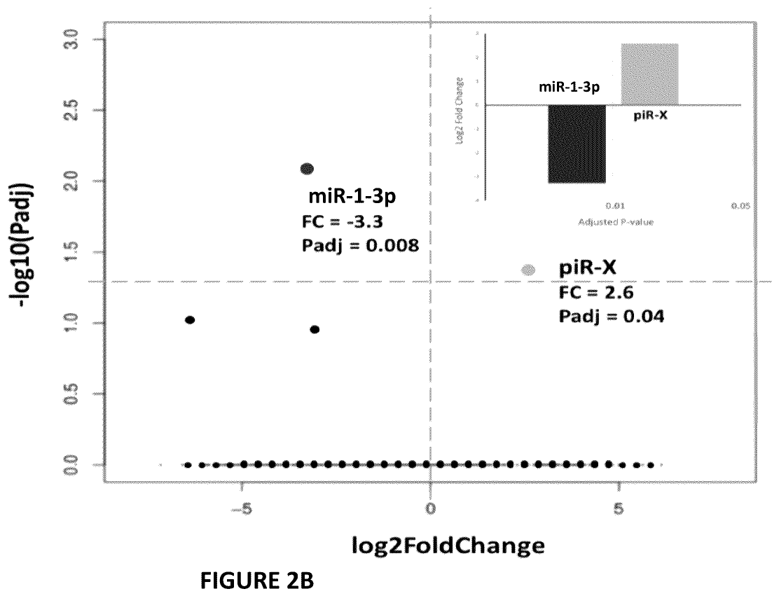

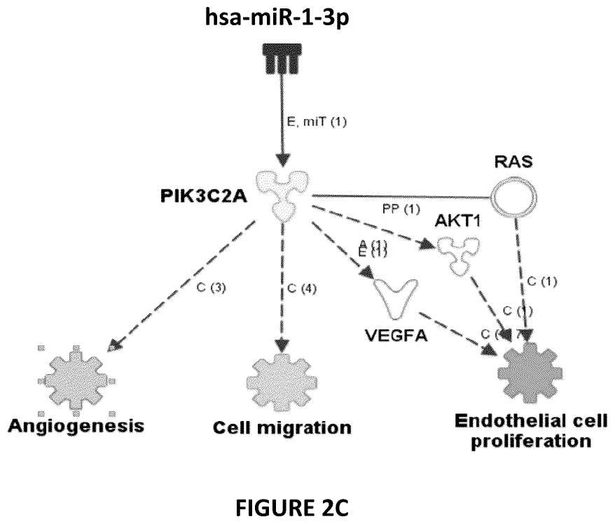

The inventors next examined differential expression of miRNAs in primary fibroblasts in diabetic compared to non-diabetic donors using next generation sequencing, RNA-Seq. Principle component analysis (PCA) showed no clustering distinction between diabetic and non-diabetic samples, which is expected due to the variability of primary human fibroblasts derived from different patients (FIG. 2A). Out of ~3000 miRNAs evaluated, the inventors found that hsa-miR-1-3p was significantly decreased (Fold Change <−2, P<0.05) (FIG. 2B). Predicted regulatory effects of hsa-miR-1-3p on different signaling pathways are presented in FIG. 2C.

### Example 4

### qRT-PCR Validation of Differentially Expressed miRNAs

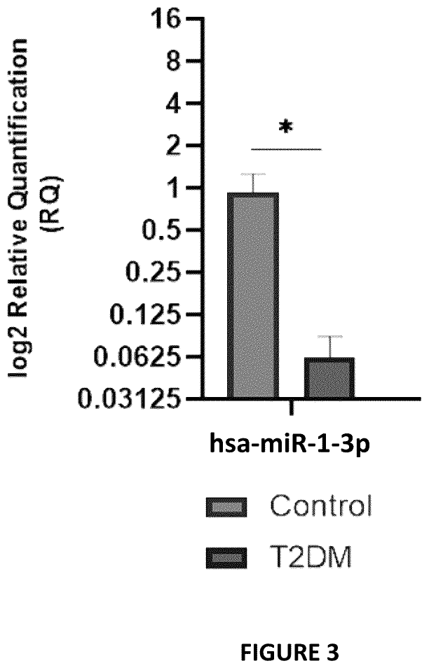

MiRNASeq results were re-examined using qRT-PCR analysis with purchased miR-X primers. The inventors observed reduced expression of hsa-miR-1-3p in diabetic fibroblasts compared to the non-diabetic fibroblasts which confirmed that hsa-miR-1-3p is downregulated in the diabetic fibroblast (FIG. 3).

### Example 5

### MiRNA Transfection Efficiency and Efficacy Evaluated by Gene Knockdown

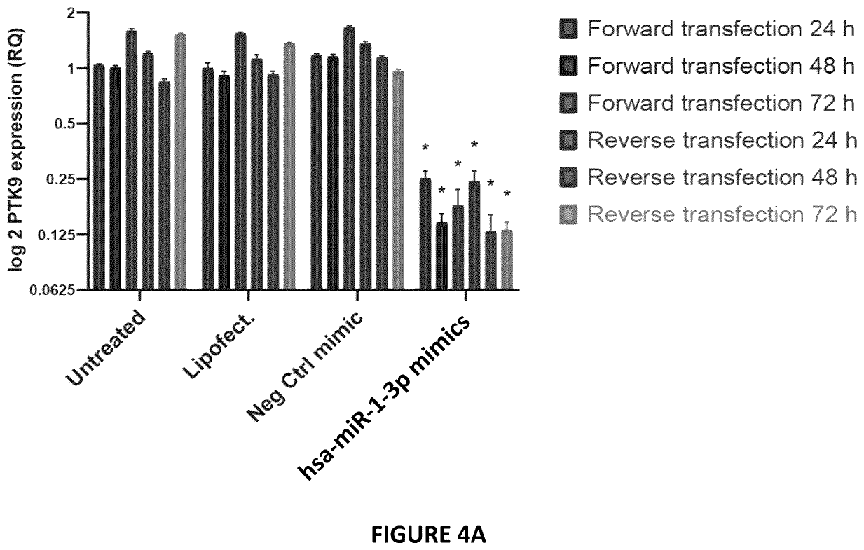

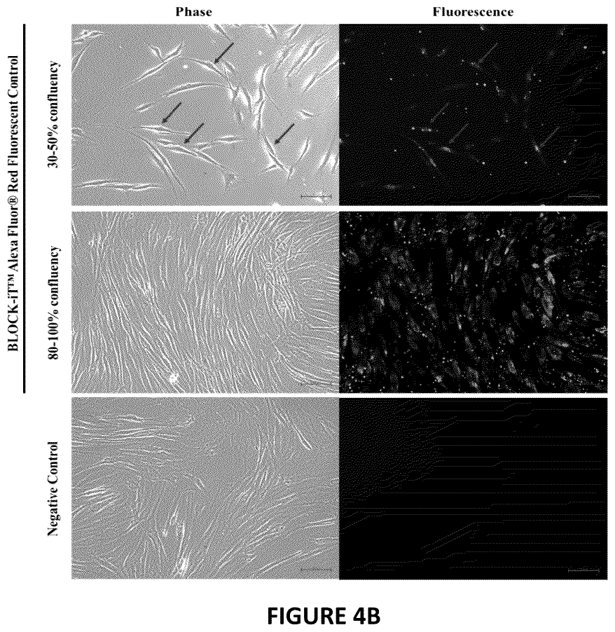

After identification of downregulated hsa-miR-1-3p in diabetic fibroblasts, diabetic fibroblasts were transfected with hsa-miR-1-3p and negative control (NC) mimics using two techniques, forward and reverse transfection, for a period of 24, 48 and 72 hours. To evaluate transfection efficacy, hsa-miR-1-3p target gene, PTK9 was quantified using qRT-PCR. Significant (P<0.001) knockdown of PTK9 was observed after 24 h in hsa-miR-1-3p transfected diabetic fibroblasts, compared to NC miRNA mimic, untreated cells and lipofectamine reagent controls. Efficacy of transfection was achieved in both techniques and continued for at least 72 h as presented in FIG. 4A.

In addition, the inventors used BLOCK-iT™ Alexa Fluor® Red Fluorescent Control as an indicator of transfection efficiency. Almost 100% of the cells picked up the oligonucleotides and cells retained a normal morphology as seen in the phase images (FIG. 4B).

### Example 6

### Diabetic Fibroblasts Expressing Hsa-miR-1-3p Display Higher Migration Rate and Secrete Increased Levels of VEGF

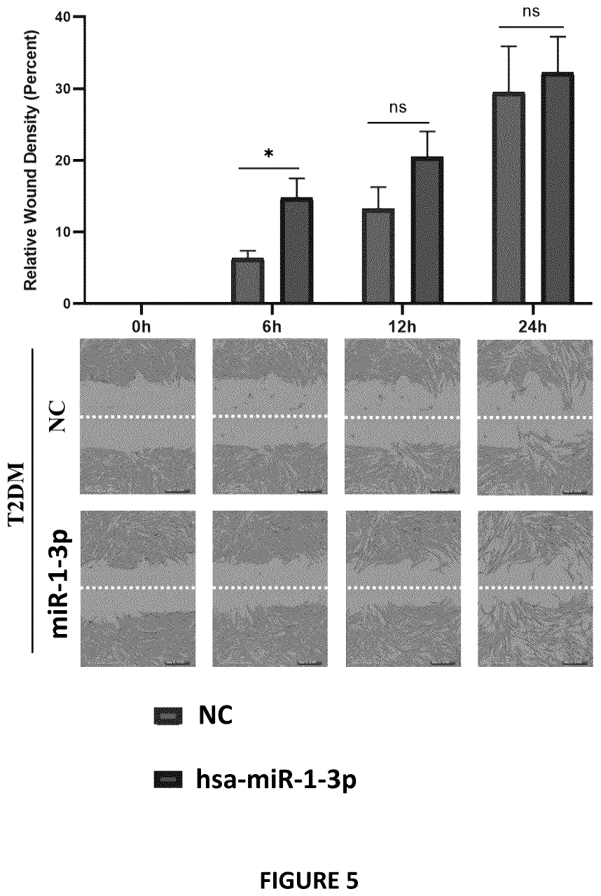

In vitro studies demonstrated that diabetic fibroblasts show selective impairments in discrete cellular processes critical for tissue repair including cellular migration. Migration of transfected diabetic fibroblasts was evaluated using a scratch assay. The data represents results of relative wound density (%) of basal diabetic fibroblasts (transfected with hsa-miR-1-3p or NC miRNA mimics) at 4 time points (FIG. 5). Diabetic fibroblasts, from 3 diabetic donors, transfected with hsa-miR-1-3p mimic demonstrated significant increase in migration ability 6 hours post scratch initiation, compared to diabetic fibroblasts transfected with NC mimic; hsa-miR-1-3p transfected fibroblasts achieved twice more closure of the wound after 6 hours (14% vs 6%, P<0.05). The wound closure in hsa-miR-1-3p transfected fibroblasts remained higher over time (12 h and 24 h).

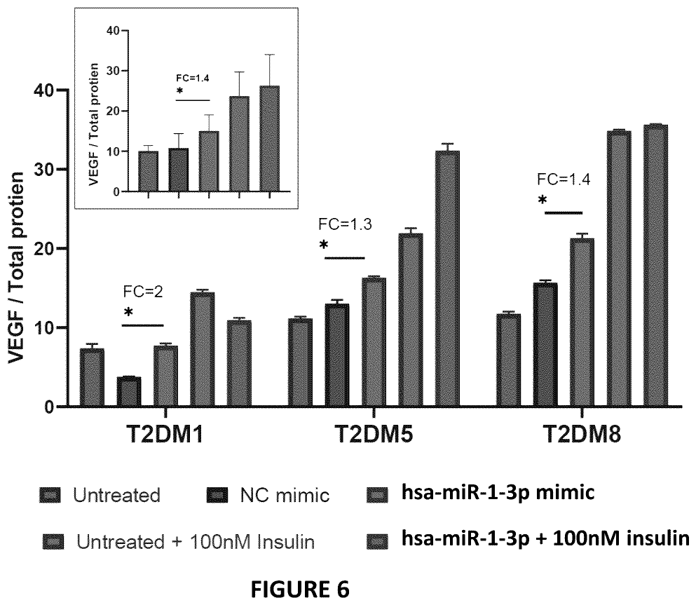

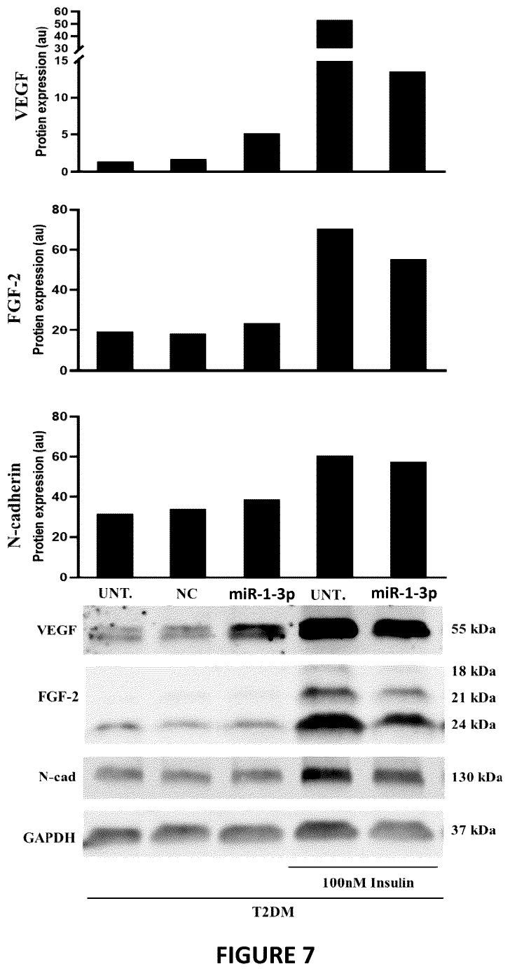

Vascular endothelial growth factor (VEGF) is involved in the angiogenesis phase of wound healing. The inventors examined basal VEGF secretion of hsa-miR-1-3p treated and NC diabetic fibroblasts (FIG. 6). The data represent significant increase in the VEGF secreted levels in the basal state of hsa-miR-1-3p-expressing diabetic fibroblasts compared to the NC mimic-expressing diabetic fibroblasts (average fold change (FC)=1.4, p<0.05). In addition, insulin treatment for 24 h increased VEGF secretion which was used as a positive control.

The inventors also examined the expression levels of factors that are secreted by fibroblasts and stimulate endothelial cell migration, such as VEGF and basic fibroblast growth factor (FGF-2), as well as factors in which their downregulation was demonstrate to accelerate cell migration, such as N-cadherin.

VEGF, FGF-2, and N-cadherin expression levels were compared between basal diabetic fibroblasts, NC miRNA and hsa-miR-1-3p transfected diabetic fibroblasts. Stimulation with 100 nM insulin served as a positive control. While no significant difference was detected in FGF-2 and N-cadherin expression between hsa-miR-1-3p transfected fibroblasts and NC, an increase in VEGF expression was demonstrated in hsa-miR-1-3p transfected fibroblasts compared to NC. In summary, expression of hsa-miR-1-3p in diabetic fibroblasts has a therapeutic potential in wound healing process, at least partially, by increasing the secretion of pro-angiogenic and pro-endothelial migration factors, such as VEGF.

### Example 7

### miR-1-3p Accelerates Wound Closure In Vivo in Diabetic Subjects

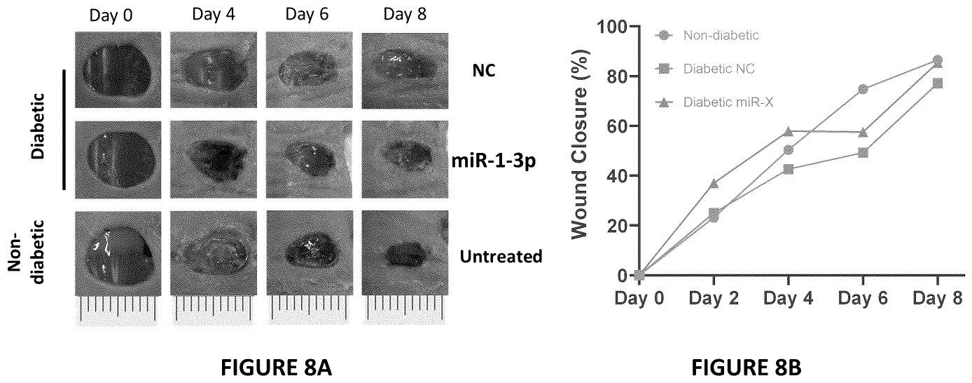

The inventors showed that miR-1-3p therapy accelerates wound healing in vivo in a diabetic murine model organism. Specifically, miR-1-3p treated diabetic mice presented wound closure rates comparable to control non-diabetic mice (FIGS. 8A-8B). Further, after 6 days of therapy, wound closure of miR-1-3p treated diabetic mice (as of control non-diabetic mice), was substantially more progressed compared to negative control administered with NC mimic miR (FIGS. 8A-8B).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method of treating a wound in a subject in need thereof, the method comprising administering to said subject a therapeutically effective amount of a pharmaceutical composition comprising an agent capable of increasing expression, abundance, or both, of a micro-RNA (miR) molecule belonging to miR-1 family in said subject, thereby treating a wound in said subject.

2. The method of claim 1, wherein said agent is selected from the group consisting of: a miR molecule, a precursor thereof, and a combination thereof.

3. The method of claim 1, wherein said pharmaceutical composition further comprises any one of: a liposome, a carrier, and both.

4. The method of claim 3, wherein any one of said: miR molecule, precursor thereof, and combination thereof, is encapsulated within said liposome.

5. The method of claim 3, wherein any one of said: miR molecule, precursor thereof, and combination thereof, is bound to said carrier.

6. The method of claim 3, wherein said carrier comprises chitosan.

7. A method for treating a wound in a subject in a need thereof, the method comprising the steps of:
(a) providing a composition comprising a fibroblast being characterized by increased expression, abundance, or both, of a miR molecule from miR-1 family; and,
(b) administering a therapeutically effective amount of said composition from step (a) to said subject,
thereby treating a wound in the subject.

8. The method of claim 7, further comprising a step preceding said step (a) comprising obtaining a fibroblast and contacting said fibroblast with an agent capable of increasing expression, abundance, or both, of a miR molecule belonging to miR-1 family, in said fibroblast.

9. The method of claim 8, wherein said fibroblast is obtained or derived from said subject.

10. The method of claim 8, wherein said subject is afflicted with diabetes.

11. The method of claim 8, wherein said agent is selected from the group consisting of: miR molecule, a precursor thereof, and a combination thereof.

12. The method of claim 7, wherein said increased is compared to a control fibroblast.

13. The method of claim 1, wherein said miR molecule belonging to miR-1 family comprises a nucleic acid sequence being selected from the group consisting of: TGGAATGTAAAGAAGTATGTAT (SEQ ID NO: 1) or a functional analog thereof having at least 80% sequence homology thereto, TGGGAAACATACTTCTTTATATGCCCATATGGACCTGCTAAGCTATGGAATGT AAAGAAGTATGTATCTCA (SEQ ID NO: 2) or a functional analog thereof having at least 80% sequence homology thereto, and ACCTACTCAGAGTACATACTTCTTTATGTACCCATATGAACATACAATGCTAT GGAATGTAAAGAAGTATGTATTTTTGGTAGGC (SEQ ID NO: 3) or a functional analog thereof having at least 80% sequence homology thereto, and any combination thereof.

14. The method of claim 1, wherein said miR molecule comprises the nucleic acid sequence set forth in SEQ ID NO: 1, or a functional analog thereof having at least 80% sequence homology thereto.

15. The method of claim 1, wherein said subject is afflicted with a disease or disorder selected from the group consisting of: metabolic disease or disorder, anemia, arterial circulatory disorder, gout, chronic venous insufficiency, rheumatoid arthritis, lupus, scleroderma, and any combination thereof.

16. The method of claim 15, wherein said metabolic disease or disorder is selected from the group consisting of: pre-diabetes, type 1 diabetes, type 2 diabetes, insulin resistance or insulin-resistance related, gestational diabetes, hyperglycemia, and any combination thereof.

17. The method of claim 1, wherein said administering comprises locally administering said pharmaceutical composition to said wound of said subject.

18.-25. (canceled)

26. A pharmaceutical composition comprising a modified fibroblast being characterized by increased expression, abundance, or both, of a miR molecule belonging to miR-1 family, compared to a control fibroblast.

27. The pharmaceutical composition of claim 26, wherein said modified fibroblast is obtained by contacting a fibroblast derived or obtained from a diabetic subject with an agent capable of increasing expression, abundance, or both, of a miR molecule belonging to miR-1 family, and optionally wherein said agent is selected from the group consisting of: miR molecule, a precursor thereof, and a combination thereof.

28.-29. (canceled)

30. The pharmaceutical composition of claim 26, being: (i) formulated for local administration, and optionally wherein said local administration comprises administration directedly to said wound of said subject or (ii) in form of: a suspension, a spray, a cream, an ointment, or any combination thereof.

31.-32. (canceled)


SEQUENCE LISTING (3 sequences)

SEQ ID NO: 1 - RNA, 22 nt, Homo sapiens
  source 1..22: mol_type = other RNA
tggaatgtaaagaagtatgtat

SEQ ID NO: 2 - RNA, 71 nt, Homo sapiens
  source 1..71: mol_type = other RNA
tgggaaacatacttctttatatgcccatatggacctgctaagctatggaatgtaaagaagtatgtatctca

SEQ ID NO: 3 - RNA, 85 nt, Homo sapiens
  source 1..85: mol_type = other RNA
acctactcagagtacatacttctttatgtacccatatgaacatacaatgctatggaatgtaaagaagtatgtatttttggtaggc